(19) United States
(12) Patent Application Publication
(10) Pub. No.: US 2019/0064160 A1
(43) Pub. Date: Feb. 28, 2019

(54) IMMUNOCHROMATOGRAPHIC DETECTION KIT

(71) Applicant: SEKISUI MEDICAL CO., LTD., Tokyo (JP)

(72) Inventors: Shinya OKUYAMA, Tokyo (JP); Kanako AZUMA, Tokyo (JP); Kazunori SAITO, Tokyo (JP)

(73) Assignee: SEKISUI MEDICAL CO., LTD., Tokyo (JP)

(21) Appl. No.: 16/095,902

(22) PCT Filed: Aug. 8, 2017
(86) PCT No.: PCT/JP2017/028679

§ 371 Date: Oct. 23, 2018

(30) Foreign Application Priority Data
Aug. 9, 2016 (JP) 2016-156287
Sep. 20, 2016 (JP) 2016-182673

(51) Int. Cl. G01N 33/559; G01N 21/64; G01N 21/78; G01N 33/543; G01N 35/00

(52) U.S. Cl. CPC G01N 33/559; G01N 21/6428; G01N 21/78; G01N 33/569; G01N 35/00029; G01N 2035/00108; G01N 33/543

(57) ABSTRACT

An object of the present invention is to provide an immunochromatographic detection kit and detection method, in which continuous increasement of coloring intensity at a detecting portion for a long time is suppressed, capturing of a complex at a detecting portion is completed within a predetermined time, and increasement of coloring intensity is stopped, when a conjugate is mixed with a liquid sample and this mixture is supplied to a test strip. The present invention addresses the problem by retaining a free antibody against the analyte antigen and a saccharide on a sample pad, separately from antibodies of the conjugate and the detecting portion, in an immunochromatographic test strip. Further, the present invention addresses the problem by retaining a specific polymeric viscous substance on a sample pad in an immunochromatographic test strip.

### TECHNICAL FIELD

The present invention relates to a detection kit including a conjugate reagent and an immunochromatographic test strip.

Particularly, the present invention relates to a detection kit wherein said test strip is a test strip in which a third antibody against an analyte and a saccharide are retained in a dissoluble manner, separately from a first antibody included in the conjugate reagent and a second antibody for immunochromatographic detection.

Furthermore, the present invention particularly relates to a detection kit wherein said test strip is a test strip in which a polymeric viscous substance is retained in a dissoluble manner on the downstream side relative to a sample-supply portion.

Further, the present invention relates to a detection method using these detection kits.

### BACKGROUND ART

The method of detecting an antigen which is an analyte (analyte antigen) in a sample using an immunochromatographic test strip is as follows. First, a conjugate in which a first antibody is immobilized to a label is brought into contact with a sample. Through this contact, the complex between analyte antigen in the sample and the conjugate is formed. The immunochromatographic test strip has a capturing portion (detecting portion) on which a second antibody is immobilized. When the above complex is provided to the carrier, the complex is captured at the capturing portion while developing in the carrier, and the presence or absence, or intensity of the complexed labeling substance may be detected by visual observation, an optical means, etc.

With regard to the mode of existence of a conjugate in detection by immunochromatography, the conjugate may exist as a conjugate part on the downstream side relative to the sample-supply portion (generally, on a sample pad) of the test strip. Alternatively, there is a mode that a conjugate may exist as a conjugate reagent such as a conjugate chip, etc., separately from the test strip (Patent Document 1). The conjugate chip is a chip in which a conjugate is applied to a filter for filtrating a sample liquid.

In the former case where the conjugate exists as a part of the test strip, when a sample is dropped to the sample-supply portion, a binding reaction of an analyte antigen in the sample with the conjugate begins from the moment the sample reaches the conjugate part downstream of the sample-supply portion. Therefore, increasement of coloring intensity at the capturing portion (detecting portion) would be stopped when all the conjugates of the conjugate part have flowed out.

In contrast, in the case where the conjugate exists as the conjugate chip, when a liquid obtained by filtrating the sample with the chip is dropped to the sample pad of the test strip, the liquid which is a mixture of the sample and the conjugate is dispersed at once throughout the sample pad. Therefore, immediately after dropping, the binding reaction of the antigen in the sample with the conjugate begins, and coloring intensity at the capturing portion (detecting portion) continues to increase until the liquid flow of the mixture reaches equilibrium.

As a method of regulating sensitivity in detection by immunochromatography, for example, Patent Document 2 is published. Patent Document 2 discloses a method of reacting an object sample for analysis with a sensitivity regulator in order to reduce analyte components. Specifically, the sensitivity regulator is an antibody labeled with an indistinguishable label (e.g., a white substance indistinguishable from the test strip), which is different from an antibody labeled with a distinguishable substance. An analysis is performed by retaining this sensitivity regulator in the upstream of an immobilization portion of the antibody labeled with a labeling substance. Part of the analyte components moves on the chromatographic carrier while reacting with the sensitivity regulator, and the analyte components that do not react with the sensitivity regulator react with the labeled body, and shall be anchored on a detecting portion.

Although this method can be expected to provide a certain effect of reducing the analyte, the mode of existence of the labeled antibody corresponds to the case where it exists as a part of the test strip as mentioned above, and there would be no problem in the first place that increase of coloring intensity continues until the flow of the liquid sample reaches equilibrium.

Further, Patent Document 3 discloses a method of detecting an analyte such as fecal occult blood by immunochromatographic detection, in which a predetermined amount of the analyte is brought into contact with an antibody immobilized on a sample pad in advance, and then unbound analyte is bound to a colored latex-conjugated antibody.

Like in Patent Document 2, in this method, the mode of existence of the labeled antibody also corresponds to the case where it exists as a part of the test strip as mentioned above, and there would be no problem in the first place that increase of coloring intensity continues until the flow of the liquid sample reaches equilibrium.

Until now, there have been no methods of suppressing long-lasting increasement of coloring intensity at a detecting portion when a mixture of a conjugate and a liquid sample is supplied to a test strip by using a conjugate chip, etc.

### CITATION LIST

### Patent Literature

Patent Document 1: International Patent Publication WO2004/081568

Patent Document 2: Japanese Patent Application Laid-Open No. H10-48210

Patent Document 3: Japanese Patent Application Laid-Open No. H08-101197

### SUMMARY OF INVENTION

### Technical Problem

An object of the present invention is to provide an immunochromatographic detection kit and detection method, in which continuous increasement of coloring intensity at a detecting portion for a long time is suppressed, capturing of a complex at a detecting portion is completed within a predetermined time, and increasement of coloring intensity is stopped, when a conjugate is mixed with a liquid sample and this mixture is supplied to a test strip by using a conjugate chip.

### Solution to Problem

In order to solve the above problem, the present inventors conducted intensive studies on how to suppress detection of an extra amount complex because a certain amount of the complex within a predetermined time is necessary for the detection of an antigen.

As a result, they thought that separately from antibodies of conjugate and detecting portions, a free antibody against the analyte antigen is allowed to exist on a sample pad to provide a mask for the antigen, thereby suppressing formation of unnecessary complexes and suppressing capturing of unnecessary complexes at the detecting portion. Further, when the free antibody singly exists on the sample pad, it is apprehended that the free antibody is immediately eluted to serve as a mask for the very complex required for detection. Therefore, the free antibody is allowed to coexist with a saccharide to provide a sustained release property and to slightly delay serving as a mask for the complex, successfully leading to selectively suppressing capture of the delayed complex, thereby completing the present invention.

That is, the present invention has the following configuration:

<1> An immunochromatographic detection kit comprising the following components:

(1) a conjugate reagent in which a first antibody immunologically reactive with an analyte is immobilized on a label; and

(2) an immunochromatographic test strip comprising an insoluble carrier which develops a sample to detect the analyte in the sample, wherein the insoluble carrier includes, in the following order from the upstream, a retaining portion on which a saccharide and a third antibody are retained in a dissoluble manner, a sample-supply portion to which the sample is supplied, and a detecting portion on which a second antibody immunologically reactive with the analyte is immobilized.

<2> The detection kit of <1>, wherein the saccharide is one or more selected from the group consisting of monosaccharides and disaccharides.

<3> The detection kit of <1> or <2>, wherein the retaining portion of the saccharide and the third antibody is formed in a line on the insoluble carrier so as to be orthogonal to the developing direction of the sample.

<4> The detection kit of any one of <1> to <3>, wherein the insoluble carrier includes at least a sample pad and a membrane pad that is different from the sample pad, the sample pad has the retaining portion of the saccharide or the third antibody and the sample-supply portion, and the membrane pad has the detecting portion.

<5> The detection kit of any one of <1> to <4>, wherein the label of (1) is a colored latex particle or a colloidal metal particle.

<6> The detection kit of any one of <1> to <5>, wherein the analyte is hemoglobin, and the first to third antibodies are anti-hemoglobin antibodies.

<7> The detection kit of any one of <1> to <6>, wherein the retaining portion of the saccharide and the third antibody is disposed on the upstream side relative to the sample-supply portion.

<8> The detection kit of any one of <1> to <7>, wherein the retaining portion of the saccharide and the third antibody is formed at the upstream end of the sample pad.

<9> An immunochromatographic detection method, the detection method comprising the following steps:

(A) mixing a conjugate reagent in which a first antibody immunologically reactive with an analyte is immobilized on a label, a specimen, and a specimen diluent to obtain a sample composed of the mixture thereof;

(B) adding dropwise the sample obtained in (A) onto a sample-supply portion of an immunochromatographic test strip described below to develop the sample in an insoluble carrier, wherein the immunochromatographic test strip comprises the insoluble carrier including, in the following order from the upstream, a retaining portion on which a saccharide and a third antibody are retained in a dissoluble manner, the sample-supply portion to which the sample is supplied, and a detecting portion on which a second antibody immunologically reactive with the analyte is immobilized; and

(C) detecting in the detecting portion a complex between the analyte in the sample and the conjugate.

<10> The detection method of <9>, wherein the saccharide is one or more selected from the group consisting of monosaccharides and disaccharides.

<11> The detection method of <9> or <10>, wherein the retaining portion of the saccharide and the third antibody is formed in a line on the insoluble carrier so as to be orthogonal to the developing direction of the sample.

<12> The detection method of any one of <9> to <11>, wherein the insoluble carrier includes at least a sample pad and a membrane pad that is different from the sample pad, the sample pad has the retaining portion of the saccharide or the third antibody and the sample-supply portion, and the membrane pad has the detecting portion.

<13> The detection method of any one of <9> to <12>, wherein the label of (1) is a colored latex particle or a colloidal metal particle.

<14> The detection method of any one of <9> to <13>, wherein the analyte is hemoglobin, and the first to third antibodies are anti-hemoglobin antibodies.

<15> The detection method of any one of <9> to <14>, wherein the retaining portion of the saccharide and the third antibody is disposed on the upstream side relative to the sample-supply portion.

<16> The detection method of any one of <9> to <15>, wherein the retaining portion of the saccharide and the third antibody is formed at the upstream end of the sample pad.

<17> The detection method of any one of <9> to <16>, wherein the specimen is feces.

It was also found that a specific polymeric viscous substance is allowed to exist on the downstream side of the sample-supply portion to suppress the flow of the antigen-bound conjugate (complex) and to suppress capturing of unnecessary complexes at the detecting portion, leading to another aspect of the present invention.

<1> An immunochromatographic detection kit comprising the following components:

(1) a conjugate reagent in which a first antibody immunologically reactive with an analyte is immobilized on a label; and

(2) an immunochromatographic test strip comprising an insoluble carrier which develops a sample to detect the analyte in the sample, wherein the insoluble carrier includes, in the following order from the upstream, a sample-supply portion to which the sample is supplied, a polymeric viscous substance-retaining portion on which a polymeric viscous substance is retained, and a detecting portion on which a second antibody immunologically reactive with the analyte is immobilized.

<2> The detection kit of <1>, wherein the polymeric viscous substance has a molecular weight of 6000 or more and a viscosity of 4 mPa·S to 1000 mPa·S.

<3> The detection kit of <1> or <2>, wherein the polymeric viscous substance-retaining portion is formed in a line on the insoluble carrier so as to be orthogonal to the developing direction of the sample.

<4> The detection kit of any one of <1> to <3>, wherein the insoluble carrier includes at least a sample pad and a membrane pad that is different from the sample pad, the sample pad has the sample-supply portion and the polymeric viscous substance-retaining portion, and the membrane pad has the detecting portion.

<5> The detection kit of <4>, wherein the polymeric viscous substance-retaining portion is disposed on the downstream side of the sample-supply portion of the sample pad.

<6> The detection kit of any one of <1> to <5>, wherein the label of (1) is a colored latex or a colloidal gold.

<7> The detection kit of any one of <1> to <6>, wherein the analyte is hemoglobin, and the first and second antibodies are anti-hemoglobin antibodies.

<8> An immunochromatographic detection method, the detection method comprising the following steps:

(A) mixing a conjugate reagent in which a first antibody immunologically reactive with an analyte is immobilized on a label, a specimen, and a specimen diluent to obtain a sample composed of a mixture thereof;

(B) adding dropwise the sample obtained in (A) onto a sample-supply portion of an immunochromatographic test strip described below to develop the sample in an insoluble carrier, wherein the immunochromatographic test strip comprises the insoluble carrier including, in the following order from the upstream, the sample-supply portion to which the sample is supplied, a polymeric viscous substance-retaining portion, and a detecting portion on which a second antibody immunologically reactive with the analyte is immobilized; and

(C) detecting in the detecting portion a complex between the analyte in the sample and the conjugate.

<9> The detection method of <8>, wherein the polymeric viscous substance has a molecular weight of 6000 or more and a viscosity of 4 mPa·S to 1000 mPa·S.

<10> The detection method of <8> or <9>, wherein the polymeric viscous substance-retaining portion is formed in a line on the insoluble carrier so as to be orthogonal to the developing direction of the sample.

<11> The detection method of any one of <8> to <10>, wherein the insoluble carrier includes at least a sample pad and a membrane pad that is different from the sample pad, the sample pad has the sample-supply portion and the polymeric viscous substance-retaining portion, and the membrane pad has the detecting portion.

<12> The detection method of <11>, wherein the polymeric viscous substance-retaining portion is disposed on the downstream side of the sample-supply portion of the sample pad.

<13> The detection method of any one of <8> to <12>, wherein the label of (1) is a colored latex or a colloidal gold.

<14> The detection method of any one of <8> to <13>, wherein the analyte is hemoglobin, and the first and second antibodies are anti-hemoglobin antibodies.

<15> The detection method of any one of <8> to <14>, wherein the specimen is feces.

### Advantageous Effects of Invention

In a method of detecting an analyte antigen in a sample, the method comprising supplying the sample, which has been brought into contact with a conjugate reagent in advance, to a sample-supply portion of an immunochromatographic test strip according to the present invention, a saccharide and a free antibody are allowed to exist in the test strip, separately from a conjugate and an antibody for detection, thereby restraining increasement of coloring intensity in a detecting portion within a predetermined period of time and enabling accurate detection in a short time.

Further, a free antibody- and saccharide-retaining portion is disposed on the upstream side in a flow direction of sample-supply portion to effectively provide a mask for the conjugate and a complex of the conjugate and the antigen that run upstream of the sample-supply portion. Therefore, it is possible to more effectively control the detection intensity of the detecting portion.

Furthermore, according to another aspect of the present invention, in a method of detecting an analyte antigen in a sample wherein the sample which has been brought into contact with a conjugate reagent in advance is supplied to a sample-supply portion of an immunochromatographic test strip, a polymeric viscous substance is allowed to exist on the downstream side relative to a sample-supply portion of the test strip, and thus inflow of excess antigens into the detecting portion is suppressed, increasement of coloring intensity in a detecting portion is restrained within a predetermined period of time, and accurate detection in a short time is enabled.

### BRIEF DESCRIPTION OF DRAWINGS

FIG. 1 is a graph showing change of color intensity over time in a test line when an antibody is maintained at a constant concentration while changing a concentration of saccharide (Example 1);

FIG. 2 is a graph showing change of color intensity over time in the test line when the saccharide is maintained at a constant concentration while changing the concentration of the antibody (Example 2);

FIG. 3 is a graph showing change of color intensity over time in the test line when only the saccharide is retained (Comparative Example);

FIG. 4 is a schematic view showing a configuration of an immunochromatographic test strip of the present invention;

FIG. 5 is a graph showing color intensity over time in a detection line when PEG 2K is retained on a sample pad;

FIG. 6 is the same as above for PEG 4K;

FIG. 7 is the same as above for PEG 6K;

FIG. 8 is the same as above for PEG 20K;

FIG. 9 is the same as above for pullulan;

FIG. 10 is the same as above for dextran 40K;

FIG. 11 is the same as above for dextran 200K;

FIG. 12 is the same as above for PVP K-25;

FIG. 13 is the same as above for PVP K-90;

FIG. 14 is the same as above for sucrose and glycerol; and

FIG. 15 is a schematic view showing a configuration of an immunochromatographic test strip of the present invention.

### DESCRIPTION OF EMBODIMENTS

(Sample)

In the present invention, a biological sample such as blood, urine, sputum, saliva, nasal discharge, other body fluids, feces, etc. may be used as a sample. The biological sample as it is may be used as the sample, or may be diluted with an appropriate diluent and filtrated and used as the sample.

(Analyte)

An analyte of the present invention may be any substance as long as it is included in a biological sample and may be detected by using an antigen-antibody reaction. Viruses, bacteria, parasites, proteins, etc. may be exemplified.

The virus as the analyte may be exemplified by influenza virus, adenovirus, rotavirus, norovirus, and the protein as the analyte may be exemplified by human hemoglobin, hepatitis B virus antibody, hepatitis C virus antibody, human immunodeficiency virus antibody, etc., in fecal occult blood.

(Kit)

An immunochromatographic detection kit of the present invention may be any detection kit including the following components of (1) and (2):

(1) a conjugate reagent in which a first antibody immunologically reactive with an analyte is immobilized on a label; and

(2) an immunochromatographic test strip comprising an insoluble carrier which develops a sample to detect the analyte in the sample, wherein the insoluble carrier includes, in the following order from the upstream, a retaining portion on which a saccharide and a third antibody are retained in a dissoluble manner, a sample-supply portion to which the sample is supplied, and a detecting portion on which a second antibody immunologically reactive with the analyte is immobilized.

Further, an immunochromatographic detection kit of another aspect of the present invention may be any detection kit including the following components of (1) and (2):

(1) a conjugate reagent in which a first antibody immunologically reactive with the analyte is immobilized on a label; and

(2) an immunochromatographic test strip comprising an insoluble carrier which develops a sample to detect the analyte in the sample, wherein the insoluble carrier includes, in the following order from the upstream, a sample-supply portion to which the sample is supplied, a polymeric viscous substance-retaining portion on which a polymeric viscous substance is retained, and a detecting portion on which a second antibody immunologically reactive with the analyte is immobilized.

These kits may include other reagents necessary for detection, a sample diluent, a test tube, a swab for fecal sampling, an instruction manual, a housing for installing the test strip, etc.

(Immunochromatographic Test Strip)

In the tests strip of the present invention, the sample-supply portion to which the sample is supplied may exist as a sample pad, separately from an insoluble membrane having the detecting portion, or may exist on the same insoluble membrane on the upstream side of the detecting portion. Of them, the sample-supply portion may preferably exist as the sample pad.

In the test strip of the present invention, a conjugate pad does not exist, and the conjugate exists as a conjugate reagent, separately from the test strip. The conjugate reagent may be, for example, in a form of a conjugate chip in which the conjugate is impregnated into a filter, or in a form of a liquid reagent in which the conjugate is added in advance to a liquid such as a specimen diluent, etc., such as a lyophilization reagent, a freezing reagent, a drying reagent, etc. The conjugate chip may be a filter itself or may be incorporated in a housing. This conjugate chip may be used to pass a specimen diluent through the filter, leading to formation of a complex of the conjugate of the chip and the analyte. The conjugate reagent in the form of the lyophilization reagent, frozen reagent, or dried reagent may be added to the analyte or the specimen diluent immediately before use.

(Sample Pad)

When the immunochromatographic test strip of the present invention includes a sample pad, the sample pad may be any pad having a portion for receiving a sample (sample-supply portion). Any substances and forms are available as long as those in a state of being molded into a pad shape may absorb a liquid sample and allow passage of the liquid and the analyte. Specific examples of materials suitable for a sample pad include, but are not limited to, glass fiber, acrylic fiber, a hydrophilic polyethylene material, dry paper, pulp, fabric, etc. Preferably, a pad made of glass fiber is used. The sample pad may include a commonly used blocking reagent for the purpose of preventing or inhibiting nonspecific reaction (adsorption) in an antibody-immobilized membrane described below.

(Saccharide and Antibody)

The saccharide to be retained in the retaining portion of the test strip of the present invention may be any saccharide as long as it has an action of controlling a dissolution rate of the free antibody, and may be preferably one or more selected from monosaccharides and disaccharides. Specifically, monosaccharides may include glucose, galactose, fructose, mannose, etc., and disaccharides may include sucrose, lactose, maltose, trehalose, fructose, etc.

The third antibody to be retained on the retaining portion of the test strip of the present invention may be any antibody as long as it is an antibody against the analyte and an antibody binding to the analyte or the complex between the analyte and the conjugate. Therefore, the third antibody may be the same as or different from the first antibody included in the conjugate or the second antibody immobilized on the detecting portion, and the third antibody may be a monoclonal antibody or a polyclonal antibody. The third antibody is preferably a free antibody which is not immobilized on the test strip and is retained in a dissoluble manner on the sample pad or the insoluble membrane, unlike an antibody bound to a solid phase such as a conjugate or an antibody immobilized on a membrane such as a detection antibody.

In the present invention, if the test strip has a sample pad, the saccharide and the antibody are preferably retained on the upstream side relative to the sample-supply portion in the sample pad.

Further, if the test strip has a sample-supply portion and a detecting portion on one and the same insoluble membrane without having a sample pad, the saccharide and the antibody are preferably retained on the upstream side relative to the sample-supply portion in the insoluble membrane. Further, the saccharide and the antibody are more preferably retained on the upstream side, particularly, the upstream end of the sample-supply portion, i.e., the upstream end of the insoluble membrane.

A method of retaining the saccharide and the antibody in a dissoluble manner on the sample pad or the insoluble membrane may be exemplified by a method of applying a solution containing the saccharide and a solution containing the antibody to the sample pad, etc., or immersing the sample pad in these solutions and drying the same pad.

Each of the solution containing the saccharide and the solution containing the antibody may be applied and dried, or a mixed solution of the saccharide and the antibody may be applied and dried.

The saccharide or the antibody may be retained in a dissoluble manner by preparing a solution containing the saccharide or the antibody at a predetermined concentration, and applying the solution to the sample pad or the insoluble membrane carrier in a line shape by using a device capable of moving a nozzle in a horizontal direction while discharging the solution from the nozzle at a constant rate, followed by drying. The concentration of the saccharide solution is preferably 0.1% to 10%, more preferably 0.5% to 8%, and much more preferably 1% to 5%. The concentration of the antibody solution is preferably 0.05 mg/mL to 5 mg/mL, more preferably 0.1 mg/mL to 1 mg/mL, and much more preferably 0.1 mg/mL to 0.7 mg/mL. Further, an amount of the saccharide or the antibody retained on the sample pad or the insoluble membrane carrier may be optimized by adjusting the discharge rate from the nozzle of the device in the case of the lateral flow type, and is preferably 1 μL/cm to 20 μL/cm, and more preferably 5 μL/cm to 10 μL/cm.

Further, the retaining portion is preferably formed in a line shape so as to be orthogonal to the flow direction, and a width of the line is preferably 1 mm to 10 mm, and more preferably 2 mm to 8 mm, and much more preferably 3 mm to 5 mm.

When a sample containing the conjugate reagent, the analyte, and the specimen diluent is supplied to the sample pad of such a test strip, the analyte and the conjugate are brought into contact with each other to form a complex while passing through the pad. The complex then develops to the insoluble membrane onto which the detection antibody is immobilized.

Since the second antibody immunologically reactive with the analyte is immobilized on a part of the insoluble membrane, the complex will be immobilized by binding to the second antibody. The immobilized complex is detected by a means for detecting absorbance, reflected light, fluorescence, magnetism, etc. which is derived from the label of the conjugate.

Here, action of the present invention will be described. Since the sample supplied to the sample-supply portion diffuses in all directions on the membrane, the sample develops downstream and some of the sample moves around upstream. Of them, some of the sample that moves around upstream develops late, which causes further increasement of coloring intensity at the detecting portion. It is likely that the saccharide and the third antibody of the present invention function to react with the sample that moves around upstream, thereby providing a mask for the analyte or the complex between the analyte and the conjugate and suppressing binding thereof to the detecting portion.

(Polymeric Viscous Substance)

The polymeric viscous substance to be retained on the test strip of the present invention may be any substance as long as it functions to aggregate the complex between the antigen in the sample and the conjugate to control the flow to the detecting portion, and preferably has a viscosity of 4 mPa·S to 1000 mPa·S. This is because a viscosity of less than 4 mPa·S lacks the ability to aggregate the complex while a viscosity of greater than 1000 mPa·S rapidly aggregates the complex, and thus it is difficult to obtain the sensitivity required at the detecting portion. Further, a molecular weight is preferably 6000 or more. More preferably, the viscosity is 4 mPa·S to 1000 mPa·S, and the molecular weight is 6000 or more. The viscosity is more preferably 5 mPa·S to 500 mPa·S, and much more preferably 10 mPa·S to 200 mPa·S. The molecular weight is more preferably 20000 or more, and much more preferably 20,000 or more to 400,000 or less. Specifically, one or more selected from polyethylene glycol (PEG), pullulan, dextran, polyvinylpyrrolidone (PVP), etc. which has a molecular weight of 6000 or more are preferred.

In the present invention, if the test strip has a sample pad, it is necessary to retain the polymeric viscous substance on the downstream side relative to the sample-supply portion in the sample pad. Further, if the test strip has a sample-supply portion and a detecting portion on one and the same insoluble membrane without having a sample pad, the polymeric viscous substance is preferably retained on the downstream side relative to the sample-supply portion in the insoluble membrane. Further, the polymeric viscous substance is more preferably retained on the downstream side relative to the sample-supply portion and on the upstream side relative to the portion overlapping with the membrane.

A method of retaining the polymeric viscous substance in a dissoluble manner on the sample pad or the insoluble membrane may be exemplified by a method of applying a solution containing the polymeric viscous substance to the sample pad, etc., or immersing the sample pad in this solution and drying the same pad.

The polymeric viscous substance may be retained in a dissoluble manner by preparing the solution containing the polymeric viscous substance at a predetermined concentration, and applying the solution to the sample pad or the insoluble membrane carrier in a line shape by using a device capable of moving a nozzle in a horizontal direction while discharging the solution from the nozzle at a constant rate, followed by drying. The concentration of the polymeric viscous substance solution is preferably controlled to the above preferred viscosity. Further, an amount of the polymeric viscous substance retained on the sample pad or the insoluble membrane carrier may be optimized by adjusting a discharge rate from the nozzle of the device in the case of the lateral flow type, and is preferably 1 μL/cm to 20 μL/cm, and more preferably 5 μL/cm to 10 μL/cm.

Further, the retaining portion is preferably formed in a line shape so as to be orthogonal to the flow direction, and a width of the line is preferably 1 mm to 10 mm, and more preferably 2 mm to 8 mm, and much more preferably 3 mm to 5 mm.

When a sample containing the conjugate reagent, the analyte, and the specimen diluent is supplied to the sample pad of such a test strip, the analyte and the conjugate are brought into contact with each other to form a complex while passing through the pad. The complex then develops to the insoluble membrane onto which the detection antibody is immobilized.

Since the second antibody immunologically reactive with the analyte is immobilized on a part of the insoluble membrane, the complex will be immobilized by binding to the second antibody. The immobilized complex is detected by a means for detecting absorbance, reflected light, fluorescence, magnetism, etc. which is derived from the label of the conjugate.

Here, action of the present invention will be described. Since the sample supplied to the sample-supply portion diffuses in all directions on the membrane, the sample develops downstream and some of the sample moves around upstream. Of them, some of the sample that moves around upstream develops late, which causes further increasement of coloring intensity at the detecting portion. It is likely that the polymeric viscous substance of the present invention functions to prevent the downstream flow of the complex between the conjugate and the sample that moves around upstream by reacting with the conjugate and the sample and aggregating the complex and to stop the downstream supply of the conjugate. That is, since the complex is captured at the polymeric viscous substance-retaining portion, only the part that passes through without being captured is bound to the detecting portion. Therefore, it is considered that binding to the detecting portion may be suppressed.

(Third Pad)

A third pad is desirably disposed as needed depending on properties, etc. of the sample, and may be any pad as long as the pad may allow passage of the complex between the analyte in the sample and the conjugate. Specifically, the third pad may include glass fiber, acrylic fiber, a hydrophilic polyethylene material, dry paper, pulp, fabric, etc., and the third pad may be particularly desirably a porous member made of polysulfone or cellulose acetate.

A pore diameter of the porous third pad of the present invention is preferably 1 μm to 100 μm, more preferably 10 μm to 100 μm, much more preferably 20 μm to 80 μm, and most preferably 25 to 70 μm in an average pore diameter. This is because a diameter of less than 1 μm causes clogging which makes an analyte flow itself poor, while a diameter of greater than 100 μm appears to make it unable to capture the nonspecific reaction substance.

(Insoluble Membrane)

The insoluble membrane used in the present invention has at least one detecting portion on which a first antibody immunologically reactive with the analyte is immobilized. The antibody immunologically reactive with the analyte may be immobilized onto an insoluble membrane carrier by a general known method. In the case of a lateral flow immunochromatographic reagent, after a solution containing a predetermined concentration of the antibody is prepared, the solution is applied to the insoluble membrane carrier in a line shape by using a device capable of moving a nozzle in a horizontal direction while discharging the solution from the nozzle at a constant rate, and is dried for immobilization. The concentration of the antibody in the solution is preferably 0.1 mg/mL to 5 mg/mL, and more preferably 0.5 mg/mL to 2 mg/mL. The amount of the antibody immobilized on the insoluble membrane carrier may be optimized by adjusting the discharge rate from the nozzle of the device in the case of the lateral flow type, and is preferably 0.5 μL/cm to 2 μL/cm.

Further, a measurement method using the lateral flow immunochromatographic reagent is a measurement method in which a sample moves in a parallel direction with respect to the insoluble membrane carrier due to capillarity.

Further, the solution containing the antibody at a predetermined concentration may be prepared by adding the antibody to a buffer solution. The kind of the buffer solution may be a commonly used buffer solution such as a phosphate buffer solution, a Tris buffer solution, and a Good's buffer solution. The buffer solution preferably has pH in a range of 6.0 to 9.5, more preferably 6.5 to 8.5, and further more preferably 7.0 to 8.0. The buffer solution may further contain salts such as sodium chloride, a stabilizer and a preservative such as sucrose, an antiseptic such as Proclin (registered trademark), etc. The salts include those contained for adjustment of ionic strength, such as sodium chloride, as well as those added for the purpose of adjustment of pH of the buffer solution, such as sodium hydroxide.

After the antibody is immobilized on the insoluble membrane, the insoluble membrane, excluding the part in which the antibody is immobilized, may be coated for blocking with a commonly used blocking agent in a form of solution or vapor.

A control capture reagent generally used for an immunochromatographic reagent may be also immobilized on the insoluble membrane. The control capture reagent, which is a reagent for ensuring the reliability of assay, captures a control reagent contained in the conjugate reagent. For example, if labeled KLH is included as a control reagent in the conjugate reagent, an anti-KLH antibody, etc. corresponds to the control capture reagent. The position at which the control capture reagent is immobilized may be appropriately selected in accordance with design of an assay system.

A membrane constituting the insoluble membrane used in the present invention may be a known membrane generally used as an insoluble membrane carrier of an immunochromatographic reagent. For example, the membrane may be made of a fiber including polyethylene, polyethylene terephthalate, nylons, glass, polysaccharide such as cellulose and cellulose derivatives, ceramics, etc. Specifically, the membrane may be glass fiber filter paper, cellulose paper, etc., commercially available from Sartorius, Millipore, Toyo Roshi, Whatman, etc. Among them, UniSart CN140 from Sartorius is preferable. By appropriately selecting a pore diameter and a structure of the insoluble membrane carrier, a flow rate of the complex between the conjugate and the analyte in the sample in the insoluble membrane carrier may be controlled.

The immunochromatographic test strip is preferably disposed on a solid phase support such as a plastic adhesive sheet. The solid phase support is made of a material not hindering the capillary flow of the sample and the conjugate. The immunochromatographic test strip may be fixed to the solid phase support with an adhesive etc. In this case, an adhesive component etc., are also made of a material not hindering the capillary flow of the sample and the conjugate. The immunochromatographic test strip may be used after being installed in or mounted on an appropriate container (housing) in consideration of the size of the immunochromatographic test strip, the addition method and the addition position of the sample, the position of formation of the detecting portion of the insoluble membrane, the signal detection method, etc., and such an installed or mounted state is referred to as a “device”.

(Antibody Immunologically Reactive with Analyte)

The first to third antibodies immunologically reactive with the analyte used in the present invention are antibodies capable of binding to the analyte. The first and second antibodies are immobilized on the label and the detecting portion described later. The third antibody, together with the saccharide, are retained in a dissoluble manner, upstream of the sample pad or on the insoluble membrane having the detecting portion.

Further, these first to third antibodies may be the same as each other, but the first and second antibodies are preferably different from each other. The antibody may be a polyclonal antibody or a monoclonal antibody. The antibody may also be a functional antibody fragment, Fab, Fab′, etc.

(Label)

As the label used in the present invention, a known label generally used in a known immunochromatographic test strip may be used. For example, the label is preferably colloidal metal particles such as gold colloid particles, platinum colloid particles, etc., colored latex particles, magnetic particles, fluorescent particles, etc., and particularly, gold colloid particles and colored latex particles are preferred.

(Conjugate Reagent)

In the conjugate used in the present invention, the antibody immunologically reactive with the analyte is immobilized to the label as described above. The method of immobilizing the antibody immunologically reactive with the analyte to the label is exemplified by physical adsorption, chemical binding, etc., and generally by physical adsorption. The conjugate of the present invention exists singly as a conjugate reagent to be mixed with the analyte, separately from the test strip. The conjugate reagent may be the conjugate as it is, or it may include components or structures other than the conjugate if necessary. For example, the conjugate reagent may be in a form of a conjugate chip in which the conjugate is impregnated into a filter. This conjugate chip may be used to filtrate the specimen diluent, leading to formation of a complex between the conjugate and the analyte.

The conjugate chip is preferably incorporated in a cap having an opening part fitted to a specimen diluent container and a nozzle. This conjugate chip is particularly called a conjugate cap. A filtration method of using the conjugate cap is as follows: the conjugate cap is fitted to the opening part of the specimen diluent container, the specimen diluent container is turned upside down to press the container, thereby passing the diluent through the conjugate chip in the conjugate cap. As a result, the diluent containing the conjugate may be discharged from the nozzle, which may be provided to the sample-supply portion of the immunochromatographic test strip.

The immunochromatographic test strip of the present invention may further include other reagents and constituents depending on the measurement conditions and the kind of the sample.

Other reagents may be exemplified by a blocking agent for preventing nonspecific reaction.

Other constituents may be exemplified by an absorbent pad that controls the developing of the sample by absorbing the sample which has moved or passed through the insoluble membrane.

The immunochromatographic test strip of the present invention may be produced in accordance with the methods described in Examples with modifications or alterations made as needed.

(Others)

In this description, the term ‘upstream or downstream’ is used to mean the upstream side or the downstream side in the flow direction of the sample. Therefore, when the test strip of the present invention has a sample pad, a third pad, and an insoluble membrane laminated from the top in a partially overlapping manner, the sample pad is on the most upstream side and the insoluble membrane is on the most downstream side. An absorbent pad (also referred to as an end pad) may be laminated on the upper side overlapping with a downstream end portion of the insoluble membrane, and in this case, the absorbent pad is on the most downstream side.

Further, in this description, the specimen diluent is also referred to as a specimen extraction liquid. The specimen diluent may also be expressed as a developing solution because it also has a function of developing the specimen.

### EXAMPLES

### [Example 1] Test for Confirming Effect of the Present Invention (1)

A saccharide and an anti-hemoglobin antibody were included at each concentration in a dissoluble manner at the upstream end of the sample pad, and the control effect on the detection reaction of hemoglobin was confirmed. Hereinbelow, a mouse monoclonal antibody was used as a colored latex-labeled anti-hemoglobin antibody, and a goat polyclonal antibody was used as the anti-hemoglobin antibody of the sample pad and of the test strip.

1. Production of Conjugate Chip

A conjugate (colored latex-labeled anti-hemoglobin antibody) and a GSA (Goat Serum Albumin) sensitized latex as a control reagent were impregnated into a sintered filter, which was then dried to produce a conjugate chip.

2. Production of Test Device

(1) Production of Sample Pad for the Present Invention

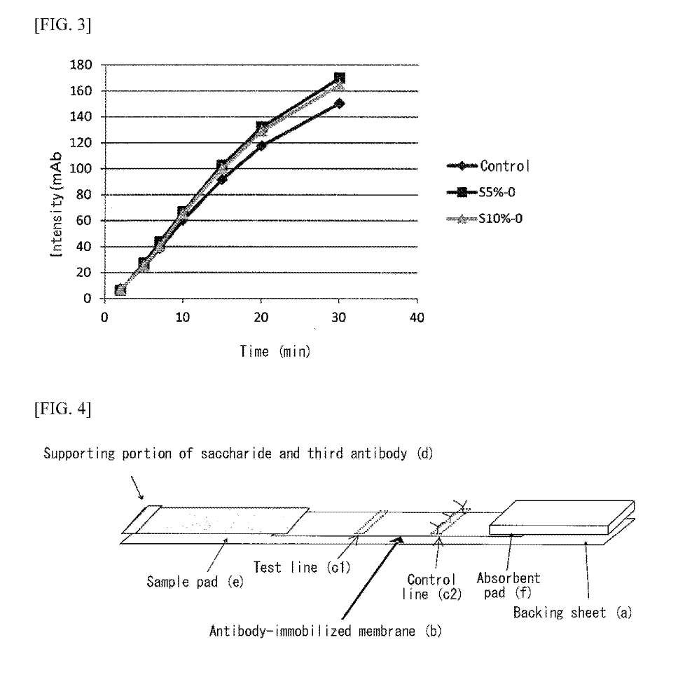

A solution containing 0% to 7% of sucrose and 0 mg/mL to 0.5 mg/mL of anti-hemoglobin antibody was applied to the surface at the end of the sample pad at 10 μL/cm, which was then dried to produce a sample pad having a retaining portion of saccharide and a third antibody for the present invention (In the figure, S1%-0.5 mg/mL indicates concentrations of the sucrose and the antibody included in the sample pad. For example, S1%-0.5 mg/mL indicates 1% sucrose and 0.5 mg/mL antibody. As shown in FIGS. 2 and 3, the control indicates the case where neither of the sucrose and antibody was included.).

(2) Production of Test Strip

FIG. 4 shows a schematic configuration of an immunochromatographic test strip of the present invention.

An antibody-immobilized membrane (b) was affixed to a plastic backing sheet (a), and a sample pad (e) for the present invention which had a retaining portion (d) of a saccharide and a third antibody produced in (1) above was mounted thereon, and an absorbent pad (f) was arranged and mounted at the opposite end thereof. In the antibody-immobilized membrane, an anti-hemoglobin antibody and a control reagent were immobilized on an insoluble membrane in the shape of lines perpendicular to the flow direction. The pads were each laminated and arranged such that the upper and lower pads were brought into contact with a portion thereof. A line composed of anti-hemoglobin antibody is referred to as a test line (c1), and a line made of a control reagent is referred to as a control line (c2).

A structure acquired by overlapping the constituent elements in this way was cut in a constant width to produce an immunochromatographic test strip.

The test strip was housed in a plastic housing having a sample-addition window and a detection window (not shown) to produce a test device.

3. Specimen Diluent Human hemoglobin was added to a specimen diluent containing 10 mM PBS to a concentration of 120 ng/mL, which was used as a measurement sample.

4. Test Method

The measurement sample was filtrated with the conjugate chip, and 5 drops thereof was dropped to the sample pad through the sample-addition window of the test device. At each time point of 2, 5, 7, 10, 15, 20 and 30 minutes after dropping, the coloring intensity of the lines was measured and quantified. Hemoglobin in the sample passes through the filter inside the conjugate chip to form a complex with the colored latex-labeled anti-hemoglobin antibody on the way of passing. The complex develops on the membrane, binds to the anti-hemoglobin antibody immobilized on the test line, and is detected.

5. Test Results

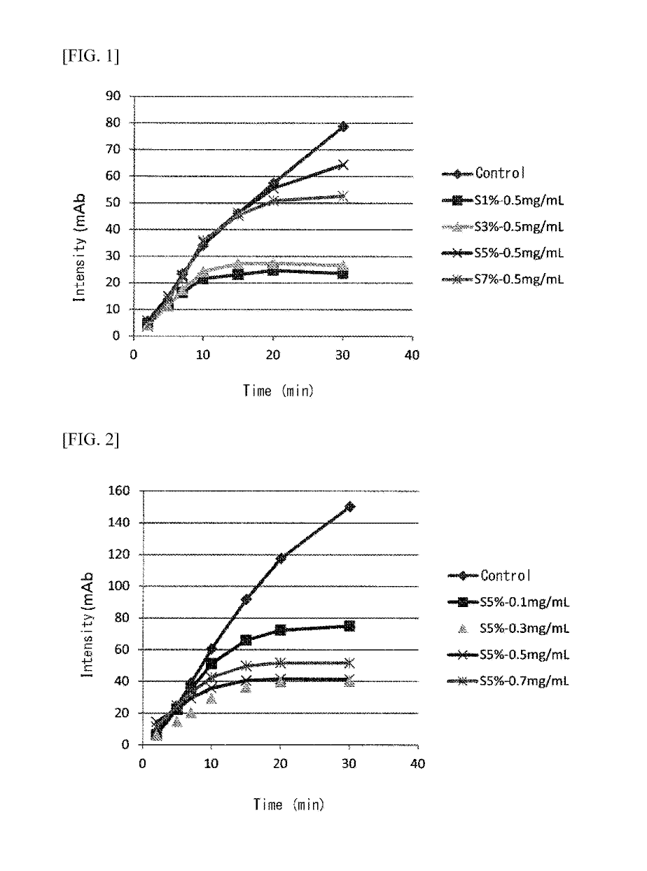

The results are shown in FIG. 1.

When a sample pad including a fixed concentration of the antibody and varying concentrations of sucrose were used, a suppressing effect on the detection reaction of hemoglobin was observed at every sucrose concentration. In particular, at the sucrose concentration of 1% to 3%, favorable results were confirmed.

### [Example 2] Test for Confirming Effect of the Present Invention (2)

A saccharide and an anti-hemoglobin antibody were included at each concentration in a dissoluble manner at the upstream end of the sample pad, and the control effect on the detection reaction of hemoglobin was confirmed.

1. Test Method

A sample pad was produced in the same manner as in Example 1, except that a solution containing 5% sucrose and 0.1 mg/mL, 0.3 mg/mL, 0.5 mg/mL, or 0.7 mg/mL of the anti-hemoglobin antibody was applied to the surface at the end of the sample pad of the test strip at 10 μL/cm, which was then dried. Thus, the test strip was produced.

2. Test Results

The results are shown in FIG. 2. When a sample pad containing a fixed concentration of sucrose (saccharide) and varying concentrations of the anti-hemoglobin antibody was used, a suppressing effect on the detection reaction of hemoglobin was observed at every antibody concentration.

### Reference Example 1

In order to compare the effects of the present invention, only saccharide was included in a dissoluble manner at the upstream end of the sample pad, and the control effect on the detection reaction of hemoglobin was confirmed.

1. Test Method

A sample pad was produced in the same manner as in Example 1, except that a solution containing 0.5% or 10% sucrose was applied to the surface at the end of the sample pad of the test strip at 10 μL/cm, which was then dried. Thus, the test strip was produced.

2. Test Results

The results are shown in FIG. 3. When a sample pad containing only sucrose (saccharide) without anti-hemoglobin antibody was used, the suppressing effect on the detection reaction of hemoglobin in the sample was not observed.

### [Example 3] Test for Confirming Effect of the Present Invention (3)

A polymeric viscous substance was included in a dissoluble manner on the downstream side of the sample-supply portion of a sample pad, and the control effect on the detection reaction of hemoglobin was confirmed. Hereinbelow, a mouse monoclonal antibody was used as a colored latex-labeled anti-hemoglobin antibody, and a goat polyclonal antibody was used as the anti-hemoglobin antibody of the sample pad and of the test strip.

1. Production of Conjugate Chip

A conjugate (colored latex-labeled anti-hemoglobin antibody) and a GSA (Goat Serum Albumin) sensitized latex as a control reagent were impregnated into a sintered filter, which was then dried to produce a conjugate chip.

2. Production of Test Device

(1) Production of Sample Pad for the Present Invention

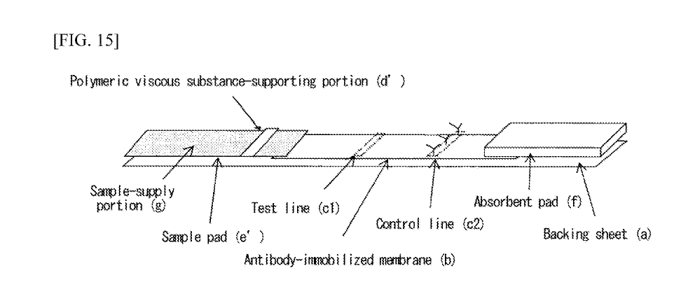

A 10 mM phosphate buffered saline solution (pH 7.2) (hereinafter, referred to as PBS solution) containing the following polymeric viscous substance at a concentration of 1%, 5%, or 10% was applied to the surface of the downstream side relative to the sample-supply portion ((g) of FIG. 15) of the sample pad at 10 μL/cm, which was then dried to produce the sample pad having a polymeric viscous substance-retaining portion for the present invention. Only the PBS solution without the polymeric viscous substance was also applied and dried to produce a sample pad. Further, a sample pad was produced by substituting the solution containing the polymeric viscous substance with sucrose (33%) or glycerol (30%). The viscosity (mPa·s) at each concentration of each of the polymeric viscous substances and polymeric substances was measured using a viscometer, and the results are shown in Table 1.

<Polymeric Viscous Substance>

PEG 2K (Wako Pure Chemical Industries, Ltd.)

PEG 4K (Wako Pure Chemical Industries, Ltd.)

PEG 6K (Wako Pure Chemical Industries, Ltd.)

PEG 20K (Wako Pure Chemical Industries, Ltd.)

Pullulan (HAYASHIBARA CO., LTD)

Dextran 40K (Wako Pure Chemical Industries, Ltd.)

Dextran 200K (Wako Pure Chemical Industries, Ltd.)

PVP K-25 (Wako Pure Chemical Industries, Ltd.)

PVP K-90 (Wako Pure Chemical Industries, Ltd.)

TABLE 1

| Tested polymer | | Visc. | M. tmp. |
|---|---|---|---|
| (Molecular weight) | Conc. | (mPa · s) | (° C.) |
| PEG 2K (2000) | 10% | 2.14 | 22.9 |
| | 5% | 1.37 | 24.0 |
| | 1% | 1.00 | 24.2 |
| PEG 4K (4000) | 10% | 2.68 | 22.6 |
| | 5% | 1.53 | 24.1 |
| | 1% | 1.03 | 24.0 |
| PEG 6K (6000) | 10% | 5.44 | 22.5 |
| | 5% | 2.43 | 24.1 |
| | 1% | 1.15 | 24.1 |
| PEG 20K (20000) | 10% | 11.40 | 23.2 |
| | 5% | 4.14 | 24.1 |
| | 1% | 1.37 | 24.0 |
| Pullulan (200000) | 10% | 86.80 | 23.5 |
| | 5% | 14.00 | 24.2 |
| | 1% | 1.86 | 23.9 |
| Dextran 40K | 10% | 3.85 | 23.5 |
| | 5% | 1.94 | 24.2 |
| | 1% | 1.11 | 24.5 |
| Dextran 200K | 10% | 13.00 | 23.1 |
| | 5% | 4.34 | 24.1 |
| | 1% | 1.37 | 24.2 |
| PVP K-25 (24000) | 10% | 4.36 | 23.1 |
| | 5% | 2.07 | 24.0 |
| | 1% | 1.12 | 24.1 |
| PVP K-90 (360000) | 10% | 160.00 | 23.1 |
| | 5% | 30.30 | 24.1 |
| | 1% | 3.23 | 25.2 |
| 33% Sucrose (342) | 33% | 3.27 | 24.1 |
| 30% Glycerol (92) | 30% | 2.08 | 24.2 |

Conc.: Concentration
Visc.: Viscosity
M. tmp.: Measurement temperature

(2) Production of Test Strip

FIG. 15 shows a schematic configuration of an immunochromatographic test strip of the present invention.

An antibody-immobilized membrane (b) was affixed to a plastic backing sheet (a), and a sample pad (e′) for the present invention which had a polymeric viscous substance-retaining portion (d′) produced in (1) above was mounted thereon, and an absorbent pad (f) was arranged and mounted at the opposite end thereof. In the antibody-immobilized membrane, an anti-hemoglobin antibody and a control reagent were immobilized on an insoluble membrane in the shape of lines perpendicular to the flow direction. The pads were each laminated and arranged such that the upper and lower pads were brought into contact with a portion thereof. The line composed of anti-hemoglobin antibody is referred to as a test line (c1), and the line made of a control reagent is referred to as a control line (c2).

A structure acquired by overlapping the constituent elements in this way was cut in a constant width to produce an immunochromatographic test strip.

The test strip was housed in a plastic housing having a sample-addition window and a detection window (not shown) to produce a test device.

3. Specimen Diluent

Human hemoglobin was added to a specimen diluent containing 10 mM PBS to a concentration of 120 ng/mL, which was used as a measurement sample.

4. Test Method

The measurement sample was filtrated with the conjugate chip, and 5 drops thereof was dropped to the sample-supply portion of the sample pad through the sample-addition window of the test device. At each time point of 2.5, 5, 7.5, 10, 15, 20, and 30 minutes after dropping, the coloring intensity of the lines was measured and quantified. Hemoglobin in the sample passes through the filter inside the conjugate chip to form a complex with the colored latex-labeled anti-hemoglobin antibody on the way of passing. The complex develops on the membrane, binds to the anti-hemoglobin antibody immobilized on the test line, and is detected.

5. Test Results and Discussion

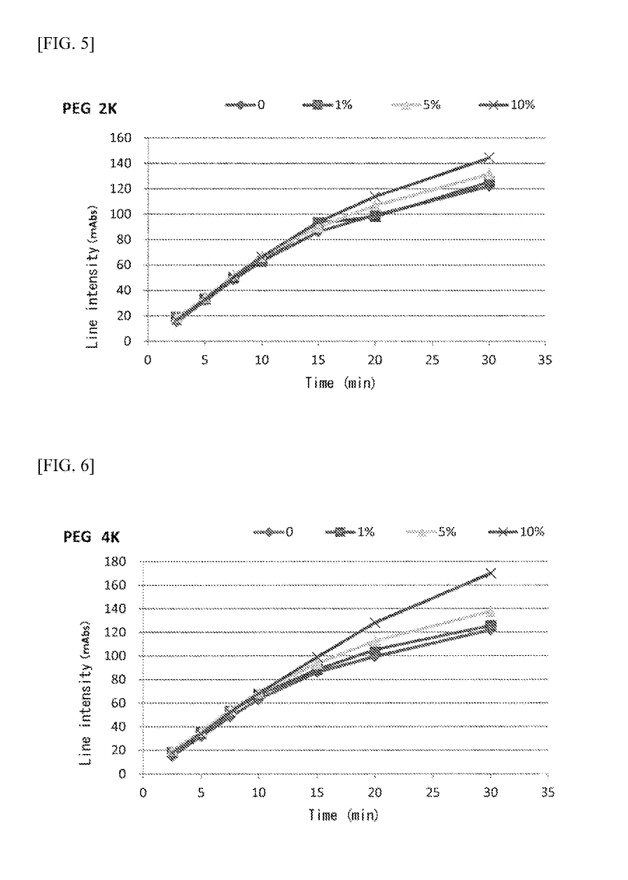

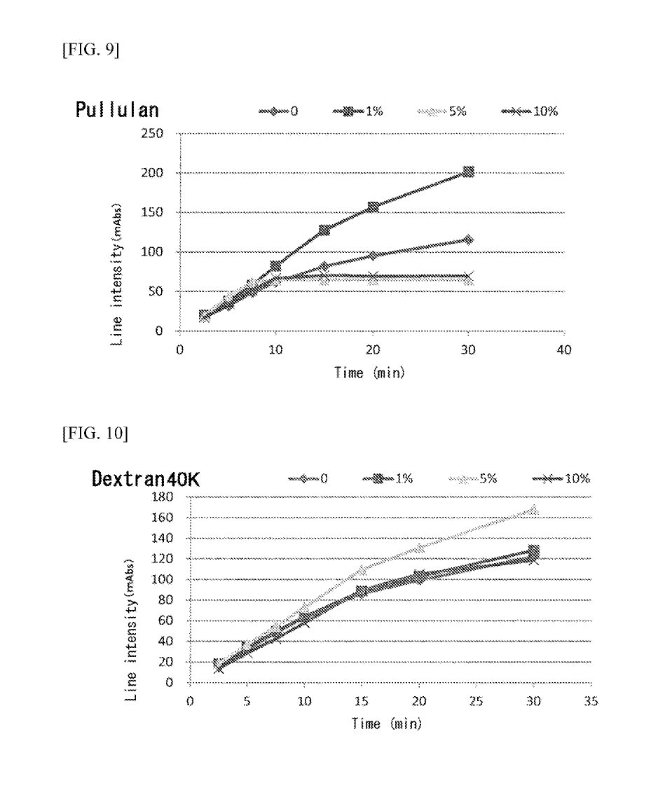

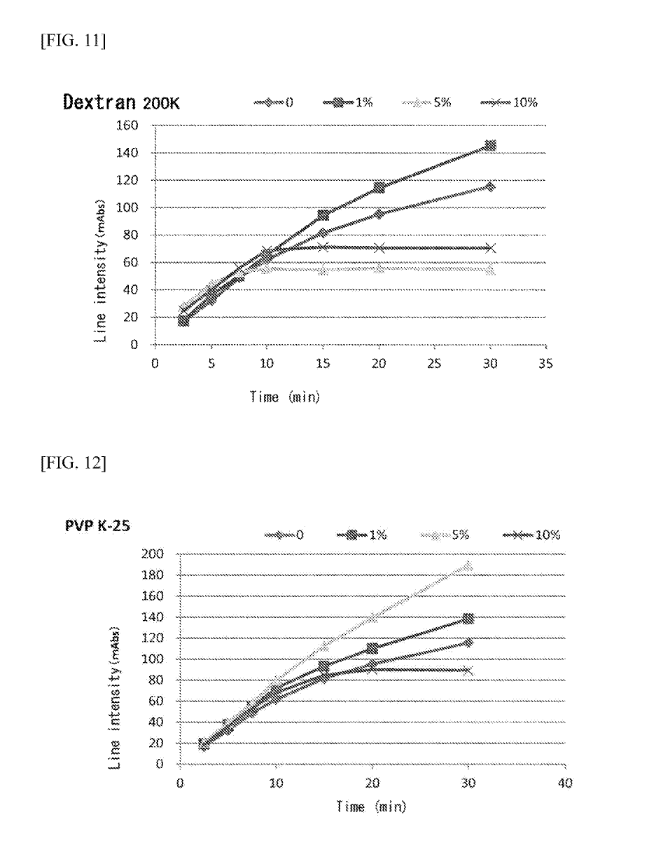

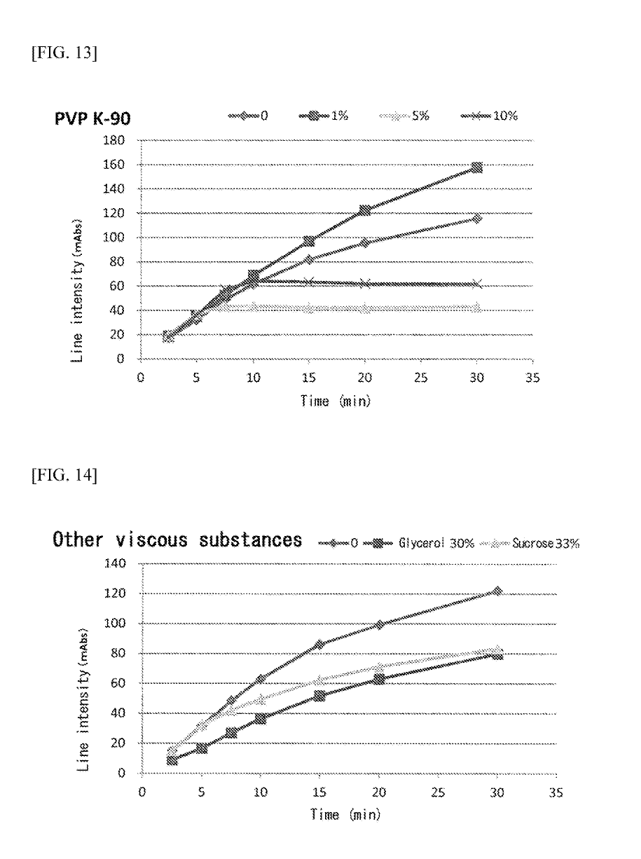

The results are shown in FIGS. 5 to 14.

As shown in FIGS. 7 and 12, a slight suppressing effect on the detection reaction of hemoglobin was observed for PEG 6K and PVP K-25 at a concentration of 10% (viscosity of 5.44 and 4.36, respectively).

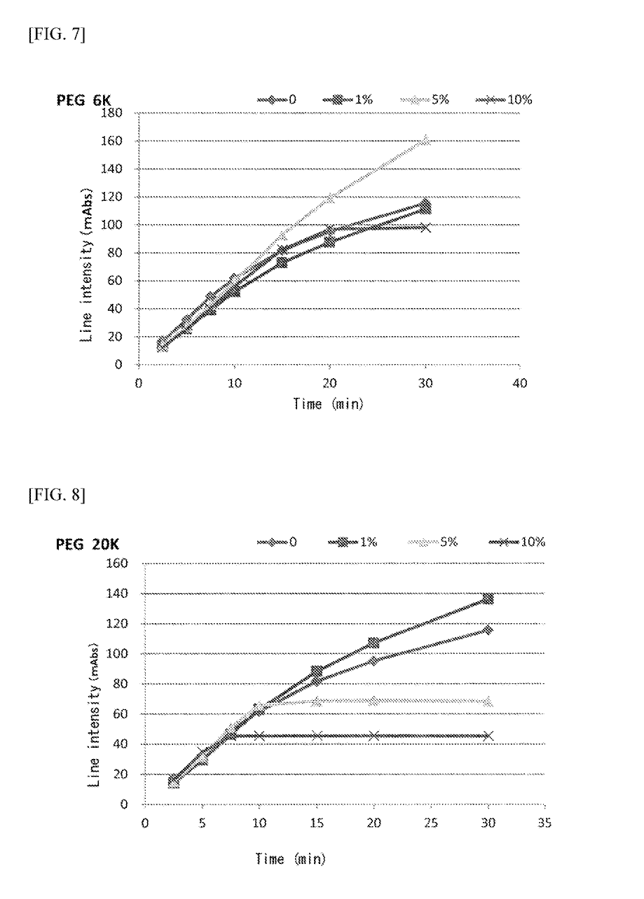

In particular, as shown in FIGS. 8, 9, 11, and 13, the suppressing effect on the detection reaction of hemoglobin was observed for PEG 20K at 5% (viscosity of 4.14) and 10% (viscosity of 11.4), pullulan at 5% (viscosity of 14.0) and 10% (viscosity of 86.8), dextran 200K at 5% (viscosity of 4.34) and 10% (viscosity of 13.0), PVP K-90 at 5% (viscosity of 30.3) and 10% (viscosity of 160.0).

In contrast, as shown in FIGS. 5, 6, and 10, the suppressing effect on the detection reaction of hemoglobin was not observed for PEG 2K, PEG 4K, and dextran 40K (viscosity of 1.0 to 2.14, 1.03 to 2.68, and 1.11 to 3.85, respectively) at every concentration.

Further, as shown in FIG. 14, the suppressing effect on the detection reaction of hemoglobin was not observed for sucrose and glycerol (viscosity of 3.27 and 2.08, respectively) at a concentration of 30% or more.

These results indicate that the polymeric viscous substances having viscosity of 4.0 or more are required in order to obtain the suppressing effect on the detection reaction of hemoglobin.

### INDUSTRIAL APPLICABILITY

In a method of detecting an analyte antigen in a sample, the method comprising supplying the sample, which has been brought into contact with a conjugate reagent in advance, to a sample-supply portion of an immunochromatographic test strip according to the present invention, a saccharide and a free antibody are allowed to exist in the test strip, separately from a conjugate and an antibody for detection, thereby restraining increasement of coloring intensity in a detecting portion within a predetermined period of time and enabling control of the reaction.

Further, in a method of detecting an analyte antigen in a sample, the method comprising supplying the sample, which has been brought into contact with a conjugate reagent in advance, to a sample-supply portion of an immunochromatographic test strip according to another aspect of the present invention, a specific polymeric viscous substance is allowed to exist on the downstream side relative to a sample-supply portion of the test strip, thereby restraining increasement of coloring intensity in a detecting portion within a predetermined period of time and enabling control of the reaction.

### REFERENCE SIGNS LIST

(a) Backing sheet
(b) Antibody-immobilized membrane
(c1) Test line
(c2) Control line
(d) Retaining portion of saccharide and third antibody
(d′) Polymeric viscous substance-retaining portion
(e), (e′) Sample pad
(f) Absorbent pad
(g) Sample-supply portion

1. An immunochromatographic detection kit, comprising the following (1) and (2a) or (2b):
(1) a conjugate reagent in which a first antibody immunologically reactive with an analyte is immobilized on a label; and
(2a) an immunochromatographic test strip comprising an insoluble carrier which develops a sample to detect the analyte in the sample, wherein the insoluble carrier includes, in the following order from the upstream, a retaining portion on which a saccharide and a third antibody are retained in a dissoluble manner, a sample-supply portion to which the sample is supplied, and a detecting portion on which a second antibody immunologically reactive with the analyte is immobilized; or
(2b) an immunochromatographic test strip comprising an insoluble carrier which develops a sample to detect the analyte in the sample, wherein the insoluble carrier includes, in the following order from the upstream, a sample-supply portion to which the sample is supplied, a polymeric viscous substance-retaining portion on which a polymeric viscous substance is retained, and a detecting portion on which a second antibody immunologically reactive with the analyte is immobilized.

2. An immunochromatographic detection kit, comprising the following components:
(1) a conjugate reagent in which a first antibody immunologically reactive with an analyte is immobilized on a label; and
(2) an immunochromatographic test strip comprising an insoluble carrier which develops a sample to detect the analyte in the sample, wherein the insoluble carrier includes, in the following order from the upstream, a retaining portion on which a saccharide and a third antibody are retained in a dissoluble manner, a sample-supply portion to which the sample is supplied, and a detecting portion on which a second antibody immunologically reactive with the analyte is immobilized.

3. The detection kit of claim 2, wherein the saccharide is one or more selected from the group consisting of monosaccharides and disaccharides.

4. The detection kit of claim 2, wherein the retaining portion of the saccharide and the third antibody is formed in a line on the insoluble carrier so as to be orthogonal to the developing direction of the sample.

5. The detection kit of claim 2, wherein the insoluble carrier includes at least a sample pad and a membrane pad that is different from the sample pad, the sample pad has the retaining portion of the saccharide or the third antibody and the sample-supply portion, and the membrane pad has the detecting portion.

6. The detection kit of claim 2, wherein the label of (1) is a colored latex particle or a colloidal metal particle.

7. The detection kit of claim 2, wherein the analyte is hemoglobin, and the first to third antibodies are anti-hemoglobin antibodies.

8. The detection kit of claim 2, wherein the retaining portion of the saccharide and the third antibody is disposed on the upstream side relative to the sample-supply portion.

9. The detection kit of claim 2, wherein the retaining portion of the saccharide and the third antibody is formed at the upstream end of the sample pad.

10. An immunochromatographic detection kit, comprising the following components:
(1) a conjugate reagent in which a first antibody immunologically reactive with an analyte is immobilized on a label; and
(2) an immunochromatographic test strip comprising an insoluble carrier which develops a sample to detect the analyte in the sample, wherein the insoluble carrier includes, in the following order from the upstream, a sample-supply portion to which the sample is supplied, a polymeric viscous substance-retaining portion on which a polymeric viscous substance is retained, and a detecting portion on which a second antibody immunologically reactive with the analyte is immobilized.

11. The detection kit of claim 10, wherein the polymeric viscous substance has a molecular weight of 6000 or more and a viscosity of 4 mPa·S to 1000 mPa·S.

12. The detection kit of claim 10, wherein the polymeric viscous substance-retaining portion is formed in a line on the insoluble carrier so as to be orthogonal to the developing direction of the sample.

13. The detection kit of claim 10, wherein the insoluble carrier includes at least a sample pad and a membrane pad that is different from the sample pad, the sample pad has the sample-supply portion and the polymeric viscous substance-retaining portion, and the membrane pad has the detecting portion.

14. The detection kit of claim 13, wherein the polymeric viscous substance-retaining portion is disposed on the downstream side of the sample-supply portion of the sample pad.

15. The detection kit of claim 10, wherein the label of (1) is a colored latex or a colloidal gold.

16. The detection kit of claim 10, wherein the analyte is hemoglobin, and the first and second antibodies are anti-hemoglobin antibodies.

17. An immunochromatographic detection method, the detection method comprising the following steps:
(A) mixing a conjugate reagent in which a first antibody immunologically reactive with an analyte is immobilized on a label, a specimen, and a specimen diluent to obtain a sample composed of a mixture thereof;
(B) adding dropwise the sample obtained in (A) onto a sample-supply portion of an immunochromatographic test strip of the following (1) or (2) to develop the sample in an insoluble carrier, wherein
the immunochromatographic test strip (1) comprises the insoluble carrier including, in the following order from the upstream, a retaining portion on which a saccharide and a third antibody are retained in a dissoluble manner, the sample-supply portion to which the sample is supplied, and a detecting portion on which a second antibody immunologically reactive with the analyte is immobilized; or
the immunochromatographic test strip (2) comprises the insoluble carrier including, in the following order from the upstream, the sample-supply portion to which the sample is supplied, a polymeric viscous substance-retaining portion, and a detecting portion on which a second antibody immunologically reactive with the analyte is immobilized; and
(C) detecting in the detecting portion a complex between the analyte in the sample and the conjugate.

18. An immunochromatographic detection method, the detection method comprising the following steps:
(A) mixing a conjugate reagent in which a first antibody immunologically reactive with an analyte is immobilized on a label, a specimen, and a specimen diluent to obtain a sample composed of the mixture thereof;
(B) adding dropwise the sample obtained in (A) onto a sample-supply portion of an immunochromatographic test strip described below to develop the sample in an insoluble carrier, wherein the immunochromatographic test strip comprises the insoluble carrier including, in the following order from the upstream, a retaining portion on which a saccharide and a third antibody are retained in a dissoluble manner, the sample-supply portion to which the sample is supplied, and a detecting portion on which a second antibody immunologically reactive with the analyte is immobilized; and
(C) detecting in the detecting portion a complex between the analyte in the sample and the conjugate.

19. The detection method of claim 18, wherein the saccharide is one or more selected from the group consisting of monosaccharides and disaccharides.

20. The detection method of claim 18, wherein the retaining portion of the saccharide and the third antibody is formed in a line on the insoluble carrier so as to be orthogonal to the developing direction of the sample.

21. The detection method of claim 18, wherein the insoluble carrier includes at least a sample pad and a membrane pad that is different from the sample pad, the sample pad has the retaining portion of the saccharide or the third antibody and the sample-supply portion, and the membrane pad has the detecting portion.

22. The detection method of claim 18, wherein the label of (1) is a colored latex particle or a colloidal metal particle.

23. The detection method of claim 18, wherein the analyte is hemoglobin, and the first to third antibodies are anti-hemoglobin antibodies.

24. The detection method of claim 18, wherein the retaining portion of the saccharide and the third antibody is disposed on the upstream side relative to the sample-supply portion.

25. The detection method of claim 18, wherein the retaining portion of the saccharide and the third antibody is formed at the upstream end of the sample pad.

26. The detection method of claim 18, wherein the specimen is feces.

27. An immunochromatographic detection method, the detection method comprising the following steps:
(A) mixing a conjugate reagent in which a first antibody immunologically reactive with an analyte is immobilized on a label, a specimen, and a specimen diluent to obtain a sample composed of a mixture thereof;
(B) adding dropwise the sample obtained in (A) onto a sample-supply portion of an immunochromatographic test strip described below to develop the sample in an insoluble carrier, wherein the immunochromatographic test strip comprises the insoluble carrier including, in the following order from the upstream, the sample-supply portion to which the sample is supplied, a polymeric viscous substance-retaining portion, and a detecting portion on which a second antibody immunologically reactive with the analyte is immobilized; and
(C) detecting in the detecting portion a complex between the analyte in the sample and the conjugate.

28. The detection method of claim 27, wherein the polymeric viscous substance has a molecular weight of 6000 or more and a viscosity of 4 mPa·S to 1000 mPa·S.

29. The detection method of claim 27, wherein the polymeric viscous substance-retaining portion is formed in a line on the insoluble carrier so as to be orthogonal to the developing direction of the sample.

30. The detection method of claim 27, wherein the insoluble carrier includes at least a sample pad and a membrane pad that is different from the sample pad, the sample pad has the sample-supply portion and the polymeric viscous substance-retaining portion, and the membrane pad has the detecting portion.

31. The detection method of claim 30, wherein the polymeric viscous substance-retaining portion is disposed on the downstream side of the sample-supply portion of the sample pad.

32. The detection method of claim 27, wherein the label of (1) is a colored latex or a colloidal gold.

33. The detection method of claim 27, wherein the analyte is hemoglobin, and the first and second antibodies are anti-hemoglobin antibodies.

34. The detection method of claim 27, wherein the specimen is feces.
